(19) United States
(12) Patent Application Publication
(10) Pub. No.: US 2013/0149277 A1
(43) Pub. Date: Jun. 13, 2013

(54) PTEROSTILBENE AND PVP GRAPE JUICE EXTRACT COMBINATION FOR TREATMENT OF METABOLIC, VASCULAR, AND NEURODEGENERATIVE DISORDERS

(71) Applicants: ChromaDex Inc., Irvine, CA (US); Cliffstar LLC, Dunkirk, NY (US)

(72) Inventors: Jeremy Bartos, Orange, CA (US); Douglas Lynch, Princeton, NJ (US)

(73) Assignees: Cliffstar LLC, Dunkirk, NY (US); ChromaDex Inc., Irvine, CA (US)

(21) Appl. No.: 13/712,747

(22) Filed: Dec. 12, 2012

Related U.S. Application Data
(60) Provisional application No. 61/570,048, filed on Dec. 13, 2011.

(51) Int. Cl. A61K 31/79; A61K 31/09

(52) U.S. Cl. CPC A61K 31/79; A61K 31/09
USPC 424/78.24

(57) ABSTRACT

A pharmaceutical composition is provided comprising a therapeutically effective amount of pterostilbene, a therapeutically effective amount of a polyphenolic-PVP extract of grape juice, and a pharmaceutically acceptable carrier. The embodiments of the pharmaceutical compositions can have lipid lowering properties, or alternatively can have properties that can treat oxidative stress, by decreasing inflammation or inflammatory processes contributing to neurodegenerative diseases. A method of lowering lipid levels in an individual is also provided, comprising administering to the individual in need of such treatment a pharmaceutical composition including a therapeutically effective amount of pterostilbene, a therapeutically effective amount of a polyphenolic-PVP extract of grape juice, and a pharmaceutically acceptable carrier, wherein lipid levels are decreased.

This application claims the benefit of earlier filed U.S. Provisional Application No. 61/570,048, filed on Dec. 13, 2011, which is hereby incorporated by reference herein.

### FIELD OF THE INVENTION

A combination of pterostilbene and a crosslinked polyphenolic-PVP grape juice extract is provided. The combination is effective for treatment of dyslipidemias, treatment of vascular disorders, and treatment of neurodegenerative disorders associated with oxidative stress.

### BACKGROUND

Pterostilbene (3,5-dimethoxy-4′-hydroxy-trans-stilbene) is a natural analog of resveratrol found in certain berries such as blueberries, cranberries, sparkleberries, lingonberries, and in very small quantities in grapes. Like resveratrol, it belongs to a class of compounds called phytoalexins, which are naturally produced by plants when under attack by pathogens such as bacteria or fungi. However, in contrast to resveratrol, animal studies have shown that pterostilbene possesses lipid and glucose lowering effects via activation of a nuclear receptor, peroxisome proliferator-activated receptor alpha isoform, PPAR-alpha (Rimando, et al., J. Agric. Food Chem. (2005) 53:3403-3407 and US2011/0060060, both incorporated herein by reference). While resveratrol exhibits only weak PPAR-alpha activation, it is known to be a PPAR-gamma activator. Of course pterostilbene displays the opposite selectively, and is only a weak PPAR-gamma activator.

With regard to aging and neurodegenerative disorders, diet supplementation with fruits and vegetables has an impact of preventing and reversing defects associated with an aging subject. Nutritional regimes that involve dietary supplementations with berryfruit (e.g., Vaccinium berries such as cranberries and blueberries) can reverse and/or forestall both motor and cognitive changes associated with aging. For example, blueberry supplementation prevents cognitive behavioral deficits in mice having increased amyloid beta-peptide production with APP/PS-1 mutations (Joseph, et al., Nutr. Neurosci. (2003) 6:153-162). The beneficial effects of berryfruits involve direct and indirect actions against oxidative stressors. As such, there is a need to identify compounds in berryfruit that are effective in altering oxidative mediated changes in motor and cognitive function. It is contemplated that individual compounds isolated from berryfruit, or combinations of such compounds with fruit extracts, may provide advantageous properties that provide useful treatment options for an individual in need thereof.

Resveratrol has been identified as having a plurality of anti-aging properties due to its robust anti-oxidant activity. In vitro experiments have shown that resveratrol is an effective free radical scavenger and inhibits low density lipoprotein oxidation (Brito, et al., Free Radic Res. (2002) 36(6):621-631). Other stilbenoids such as pinostilbene, desoxyrhapontigenin, pterostilbene, resveratrol trimethylether, and piceatannol, have varying degrees of biological activity and effectiveness for lowering lipid levels by activating PPAR-alpha.

As discussed above, pterostilbene, a natural methylether analog of resveratrol, has been demonstrated to have antioxidant activity similar to that of resveratrol (Rimando, et al., J. Agric. Food Chem. (2002) 50:3453-3457; Stivala, et al., J. Biol. Chem. (2001) 276(25):22586-22594). Pterostilbene is present in some small fruits such as grapes (Adrian, et al., J. Agric. Food Chem. (2000) 48:6103-6105) and berries of Vaccinium (Vaccinium ashei Reade and Vaccinium stamineum L.) (Rimando, et al., J. Agric. Food Chem. (2004) 52:4713-4719) as well as in woody plants (Maurya, et al., J. Nat. Prod. (1977) 47:179-181; Amone, et al., J. Chem. Soc. Perkins Trans. (1977) 19:2116-2118). However, Rimando, et al. (2004) found that in grapes, pterostilbene is generally found in very minute quantities and may be completely absent. Additionally, a plurality of botanicals contain pterostilbene, including Anogeissus acuminata, Dracaena cochinchinensis, Dracaena loureiri, Guibourtia tessmannii, Pterocarpus macrocarpus, Pterocarpus marsupium, Pterocarpus santalinus, Vaccinium ashei, Vaccinium corymbosum, Vaccinium deliciosum, Vaccinium membranaceum, Vaccinium ovatum, Vaccinium ovalifoilum, Vaccinium parviflorum, Vaccinium stamineum, Vaccinium uliginosum, and Vitis vinifrea. Pterostilbene is also found in non-botanical sources such as propolis.

Blueberry species vary in the amount of pterostilbene concentration. It has been reported that a range of 99 ng to 475 ng of pterostilbene can be derived from one gram of lyophilized blueberries.

Studies have linked the effects of antioxidants with deleterious effects of brain aging and behavior. The combination of antioxidant/anti-inflammatory polyphenolics including pterostilbene found in fruits and vegetables in the form of “secondary chemicals” not generally involved in the plant primary metabolism, has exhibited efficacy in preventing these deleterious effects. As such, there is a need to further identify fruit and plants, specifically the compounds that can protect against aging and cognitive defects. See, US2009/0069444, incorporated herein by reference.

Further, it is known that factors such as life style and diet can negatively or positively influence the outcome of vascular disease. Exercise and diet can significantly decrease the rate and extent of plaque formation. It also appears that diet can strongly influence the likelihood that existing plaque will result in serious blood clots. Therefore, it is not surprising that compounds in a number of foods mimic the anticoagulation and anti-platelet aggregation caused by drug treatments. Recent studies have demonstrated anti-thrombotic and anti-platelet properties in a variety of foods including berries and grapes.

Polyphenolic compounds found in grapes may have effects on human blood cells, thus affecting platelet aggregation and/or blood coagulation. Plant extracts can be produced by treating plant or fruit juices including grape juice with binding materials such as crosslinked polyvinyl pyrrolidone (PVP) and cholestyramine, as described in US2010/0233672, incorporated herein by reference.

In view of the above, it would be desirable to provide a pharmaceutical composition having the beneficial properties of pterostilbene and a fruit juice extract.

If a way could be found to enhance the lipid lowering properties of pterostilbene by combination with a readily available polyphenolic-PVP extract of grape juice, this would represent a valuable contribution to the art.

Furthermore, if a way could be found to decrease inflammatory processes contributing to neurodegenerative diseases and aging in an individual by combining pterostilbene with a readily available polyphenolic-PVP extract of grape juice, this would represent a valuable contribution to the art.

### SUMMARY OF THE INVENTION

A pharmaceutical composition is provided comprising a therapeutically effective amount of pterostilbene, a therapeutically effective amount of a polyphenolic-PVP extract of grape juice, and a pharmaceutically acceptable carrier. The embodiments of the pharmaceutical compositions can have lipid lowering properties. In another embodiment, a pharmaceutical composition comprises a therapeutically effective amount of a combination of pterostilbene and a polyphenolic-PVP grape juice extract.

In alternative embodiments, the pharmaceutical compositions can have properties that can treat oxidative stress, by, for example, decreasing inflammation or inflammatory processes contributing to neurodegenerative diseases.

A method of lowering lipid levels in an individual is provided, comprising administering to the individual in need of such treatment a pharmaceutical composition including a therapeutically effective amount of pterostilbene, a therapeutically effective amount of a polyphenolic-PVP extract of grape juice, and a pharmaceutically acceptable carrier, wherein lipid levels are decreased.

A method of lowering low-density lipoprotein cholesterol (LDL-C) and raising high-density lipoprotein cholesterol (HDL-C) in an individual is provided, comprising administering to the individual in need of such treatment a pharmaceutical composition comprising a therapeutically effective amount of pterostilbene, a therapeutically effective amount of a polyphenolic-PVP extract of grape juice, and a pharmaceutically acceptable carrier, wherein (LDL-C) levels are decreased, and wherein (HDL-C) levels are increased.

A method of lowering blood pressure in an individual is provided, comprising administering to the individual in need of such treatment a pharmaceutical composition comprising a therapeutically effective amount of pterostilbene, a therapeutically effective amount of a polyphenolic-PVP extract of grape juice, and a pharmaceutically acceptable carrier, wherein systolic blood pressure (SBP) or diastolic blood pressure (DBP) is decreased.

### BRIEF DESCRIPTION OF THE DRAWINGS

FIG. 1 depicts total LDL-C (mg/dL) in one embodiment showing the effects of: pterostilbene (high dose), and combinations of pterostilbene (low dose) with and without a polyphenolic-PVP grape juice extract. Dark solid bar: initial baseline measurement; light solid bar: final measurement after 6-8 weeks of treatment.

FIG. 1A depicts total LDL-C (mg/dL) in one embodiment showing the effects of combinations of pterostilbene (low dose) and a polyphenolic-PVP grape juice extract, with and without a statin. Dark solid bar: initial baseline measurement; light solid bar: final measurement after 6-8 weeks of treatment.

FIG. 2 depicts total cholesterol (TC in mg/dL) in one embodiment showing the effects of: pterostilbene (high dose), and combinations of pterostilbene (low dose) with and without a polyphenolic-PVP grape juice extract. Dark solid bar: initial baseline measurement; light solid bar: final measurement after 6-8 weeks of treatment.

FIG. 2A depicts total cholesterol (TC in mg/dL) in one embodiment showing the effects of combinations of pterostilbene (low dose) and a polyphenolic-PVP grape juice extract, with and without a statin. Dark solid bar: initial baseline measurement; light solid bar: final measurement after 6-8 weeks of treatment.

### DETAILED DESCRIPTION

A safe and effective pharmaceutical or nutraceutical composition has been provided containing pterostilbene and a polyphenolic-PVP extract of grape juice. In one embodiment, a method of treating an individual for a dyslipidemia comprises the step of administering to the individual in need of such treatment a pharmaceutical composition comprising a therapeutically effective amount of pterostilbene, a therapeutically effective amount of a polyphenolic-PVP extract of grape juice, and a pharmaceutically acceptable carrier.

In another embodiment, a method of lowering lipid levels in an individual comprises the step of administering to the individual in need of such treatment a pharmaceutical composition comprising a therapeutically effective amount of pterostilbene, a therapeutically effective amount of a polyphenolic-PVP extract of grape juice, and a pharmaceutically acceptable carrier, wherein lipid levels are decreased.

The effects of pterostilbene on PPAR-alpha activation are well-established. The peroxisome proliferator-activated receptor (PPAR) isoforms belong to the nuclear receptor superfamily of ligand-activated transcription factors which control gene expression by interacting with specific response elements in the promoter region of target genes. The primary isoform involved in fatty acid and lipid catabolism and in the activation of genes involved in fatty acid oxidation in the liver is PPAR-alpha. It has been shown that activation of PPAR-alpha in the liver leads to increased oxidation of fatty acids as well as decreased triglyceride and very low density lipoprotein (VLDL) synthesis (Fruchart, et al., Ann. Pharm. Fr. (2004) 62:3). This activation, coupled with its ability to induce hepatic apoliporotein A-I and A-II expression, leading to increased plasma HDL cholesterol, makes it a very important target in the cholesterol-lowering field (Gervois, et al., Clin. Chem. Lab. Med. (2000) 38:3). Comparatively, the fibrate family of pharmaceuticals are known PPAR-alpha agonists, and their triglyceride lowering and HDL-cholesterol raising effects are mainly attributed to their activation of PPAR-alpha (Desai, et al., Bioorg. Med. Chem. Lett. (2006) 16:1673).

Dyslipidemias are disorders of lipoprotein metabolism, including lipoprotein overproduction or deficiency. These disorders may be manifested by elevation of the serum total cholesterol (TC), low-density lipoprotein (LDL) cholesterol and triglyceride concentrations, and a decrease in the high-density lipoprotein (HDL) cholesterol concentration. Very low-density lipoprotein (VLDL) and total lipoprotein may also be affected.

In certain embodiments of the present invention, the decreased lipid levels may be expressed as a reduction in blood plasma or serum selected from total cholesterol (TC), low-density lipoprotein cholesterol (LDL-C), triglycerides (TG), and the ratio of low-density lipoprotein cholesterol to high-density lipoprotein cholesterol (LDL-C/HDL-C). Measurement and diagnostic determination of blood levels of the aforementioned lipid components in various animal species, mammals, and human individuals is well known in the art.

In other embodiments, blood levels of high-density lipoprotein cholesterol (HDL-C) may be increased using the compositions disclosed herein.

The UDP-glucuronosyltransferases (UGTs) catalyze the transfer of glucuronic acid from a high-energy cofactor, UDP-glucuronic acid, to a xenobiotic, drug, or endogenous substrate containing an available reactive or nucleophilic center such as a hydroxyl, carboxyl, amino, or thiol group. The UGTs are Phase II biotransformation enzymes predominantly expressed in liver and intestine, and are membrane-bound enzymes localized on the luminal surface of the endoplasmic reticulum. Relative to the parent substrate, the end-products of glucuronidation are typically more polar and better suited for excretion and elimination through the urine or bile.

Endogenous UGT substrates include bilirubin, neutral steroids, bile acids, fatty acids, and retinoids. Xenobiotic UGT substrates range from environmental toxicants such as benzo[a]pyrene to common pharmaceuticals such as acetaminophen and other NSAIDs. Individual UGT isoforms display distinctive patterns of substrate specificity and inducible regulation. Different UGTs are expressed in a species- and tissue-specific manner. The two major UGT gene families are UGT1 and UGT2.

PPARs are known to be involved in the regulation of UGTs. In particular, the UGTs are targets of PPAR-alpha, as opposed to PPAR-gamma. Resveratrol, a known PPAR-gamma activator, has shown some induction of UGTs, but is not a strong activator of PPAR-alpha. In one study of a group of similar stilbenes, only pterostilbene activated PPAR-alpha, in a manner similar to ciprofibrate (Rimando, et al., J. Agric. Food Chem. (2005) 53:3403-3407). Pterostilbene is not thought to be a strong activator of PPAR-gamma. This sub-type selectivity is thought to be advantageous in the present approach to activate or induce expression of UGTs using pterostilbene.

In addition, pterostilbene can be useful in the treatment or prevention of vascular disease. Pterostilbene has been shown to inhibit the proliferation of rat smooth muscle cells in vitro which gives it potential as an anti-proliferative agent for the treatment of atherosclerosis (Park, et al., Vascul. Pharmacol. (2010) 53: 61). In an embodiment, a combination of a therapeutically effective amount of pterostilbene and a therapeutically effective amount of a polyphenolic-PVP grape juice extract can be used for treating atherosclerosis. It is expected that the combination of pterostilbene and the polyphenolic-PVP grape juice extract will provide synergistic effects with regard to treating atherosclerosis.

The presence of chronic inflammatory conditions in the colonic environment has been implicated in the development of colorectal cancer, and treatment regimens against inflammatory markers have reduced the risk of colon cancer. Pterostilbene has been shown to reduce the levels of pro-inflammatory cytokine markers such as COX-2, TNF-alpha, IL-4, IL-1-beta, and iNOS both in vitro and in vivo. (See, for example, Hougee, et al., Planta Med. (2005) 71(5):387-392; Paul, et al., Cancer Prev. Res. (Phila). (2009) 2(7):650-657; and Paul, et al., Carcinogenesis (2010) 31(7): 1272-1278, each incorporated herein by reference.)

In another embodiment, a combination of a therapeutically effective amount of pterostilbene and a therapeutically effective amount of a polyphenolic-PVP grape juice extract can be used for reducing the levels of a pro-inflammatory cytokine. It is expected that the combination of pterostilbene and the polyphenolic-PVP grape juice extract will provide synergistic effects with regard to treating, preventing, and/or reducing inflammation.

Pterostilbene (3,5-dimethoxy-4′-hydroxy-trans-stilbene) is an orally bioavailable compound with a half life t_{1/2} of about 74-105 minutes in blood. In contrast, resveratrol has poor bioavailability, and is readily metabolized by UGTs leading to a much shorter half life (t_{1/2} about 10-14 minutes in blood), which hinders its effectiveness as a chemopreventive agent.

Pterostilbene (99% purity) is commercially available from ChromaDex, Inc. (Irvine, Calif.).

In one embodiment, pterostilbene can be provided in daily dosages of from about 10 mg to about 500 mg, in particular in a human patient, for example. Another suitable dosage range is from about 25 mg to about 500 mg daily. Another suitable dosage range is from about 50 mg to about 250 mg daily. Another suitable dosage range is from about 50 mg to about 150 mg daily. Another suitable dosage range is from about 50 mg to about 100 mg daily. A particularly suitable dosage is about 100 mg administered daily.

Extracts of grapes, grape juice, or other berries can be used in the embodiments of the present invention. Useful berries can include Vaccinium spp. such as, for example, blueberries, cranberries, sparkleberries, lingonberries, deerberries, bilberries, partridgeberries, and the like. Optionally, extracts or blends derived from berries can be used. In turn, the extracts and/or blends can be further blended with the pharmaceutical or nutraceutical compositions disclosed herein. Many commercially available berry products are readily available.

A useful extract is grape juice extract that has been bound to crosslinked polyvinyl pyrrolidone (PVP) or ion exchange resin. One particularly useful grape juice extract is a polyphenolic-PVP extract granular powder sold as ShanStar™ Concord Grape Extract (Star PhytoNutrients Division of Cott Beverages, Inc., Dunkirk, N.Y.). These useful polyphenolic-PVP extracts of grape juice can have a high polyphenolic content, in a range from about 200 mg/g to about 250 mg/g.

Representative procedures for preparation of the polyphenolic-PVP extracts of grape juice can be found in US2010/0233672, incorporated herein by reference in its entirety.

In one embodiment, the polyphenolic-PVP grape juice extract can be used in amounts ranging from about 50 mg to about 500 mg, in particular in a human patient, for example. Another suitable dosage range is from about 100 mg to about 400 mg daily. Another suitable dosage range is from about 100 mg to about 300 mg daily. Another suitable dosage range is from about 150 mg to about 300 mg daily. Another suitable dosage range is from about 200 mg to about 300 mg daily. A particularly suitable dosage is about 200 mg administered daily.

Without being bound by theory, it is believed that, since grapes contain very little pterostilbene, as discussed above, a composition including a combination of pterostilbene and grape extract components may provide advantageous properties, and/or unexpected advantages to a patient, above the value of their independent administration.

The dosages of the polyphenolic-PVP grape juice extract are expected to be particularly effective when used in combination with pterostilbene in solid or liquid form, or as a blend. It is expected that the combination of pterostilbene and the polyphenolic-PVP grape juice extract will provide synergistic effects with regard to lipid lowering and reduction of biological markers of oxidative stress.

In another embodiment, a pharmaceutical composition comprises a therapeutically effective amount of a combination of pterostilbene and a polyphenolic-PVP grape juice extract. Due to expected synergy of the components, the pharmaceutical composition of the combination of pterostilbene and a polyphenolic-PVP grape juice extract may provide a therapeutic effect even where the individual components may not provide the therapeutic effect.

A composition in accordance with the present invention containing pterostilbene, or derivatives thereof, or a pharmaceutically acceptable salt of pterostilbene, or a co-crystallization of pterostilbene and one other compound, or an emulsified version of pterostilbene, can be prepared by conventional procedures for blending and mixing compounds. In another embodiment, pterostilbene in combination with a polyphenolic-PVP grape juice extract can be prepared by conventional procedures for blending and mixing compounds. Preferably, the composition also includes an excipient, most preferably a pharmaceutical excipient. Compositions containing an excipient and incorporating the pterostilbene can be prepared by procedures known in the art. Optionally, the composition can include one or more adjuvants, excipients, carriers, buffers, diluents, and/or other customary pharmaceutical auxiliaries. For example, pterostilbene can be formulated into tablets, capsules, powders, suspensions, solutions for oral administration and solutions for parenteral administration including intravenous, intradermal, intramuscular, and subcutaneous administration, and into solutions for application onto patches for transdermal application with common and conventional carriers, binders, diluents, and excipients.

The nutraceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user. Useful excipients include, but are not limited to, microcrystalline cellulose, magnesium stearate, calcium stearate, any acceptable sugar (e.g., mannitol, xylitol), and the like, and for cosmetic use an oil-base is preferred.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, pterostilbene and/or pterostilbene in combination with a polyphenolic-PVP grape juice extract may be further combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably may contain from five or ten to about seventy percent of the active compound. Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g., by injection, for example bolus injection or continuous infusion) and may be presented in unit dose for administration in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Routes of Administration

The compounds may be administered by any route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

Without intending to be bound by theory, pterostilbene dosage can be estimated or translated from dosages used in animal studies. Doses from animal studies were translated to human doses by utilizing a Kₘ factor ratio, where Kₘ factors were assigned to each animal model based on their body surface area (Reagan-Shaw, et al., FASEB J. (2007) 22:659-661). The human equivalent dose (HED) is equal to the animal dose multiplied by the ratio animal's Kₘ/human Kₘ. For example, in the hypercholesterolemic hamster cholesterol study, the hamsters were given 2.5 mg/kg of pterostilbene per day (Rimando et al., 2005). With a Kₘ ratio of 0.135 (with Kₘ values of 5 for hamster/37 for human), the human equivalent dose for this study is 0.337 mg/kg for an adult human, or approx. 25 mg for a 160 pound person per day. Additional studies on diabetes and cognitive function in rats have given human equivalent doses of 118-471 mg/day and 30-118 mg/day respectively (Pari, et al., Life Sci. (2006) 79:641; Joseph, et al., J. Agric. Food Chem. (2008) 56:10544). A pterostilbene 28-day subacute toxicity study in mice showed no local or systemic toxicity at 160 pound human equivalent doses of approximately 125 mg/day, 1.25 g/day, and 12.5 g/day (Ruiz, et al., J. Agric. Food Chem. (2009) 57:3180). Each reference is incorporated herein by reference.

The following is a general study design for human clinical trial to assess efficacy of pterostilbene to lower cholesterol and/or lower blood pressure, among other measured endpoints including markers for oxidative stress. The study type is interventional: a randomized, double-blind, placebo controlled study. Endpoint Classification: Safety/Efficacy Study.

Purpose

The purpose of this study is to evaluate whether pterostilbene alone, and a combination of pterostilbene and a polyphenolic-PVP extract of grape juice, will help control cholesterol and blood pressure, as well as improve markers for oxidative stress in patients with dyslipidemia meeting inclusion criteria. The investigators will assess the safety of pterostilbene in these patients.

Study Population

Eighty subjects, divided into four groups: 20 per study group. Ages eligible for study: 18 to 88 years, both genders. No healthy volunteers accepted. Inclusion criteria: patients≧18 years of age with a previous TC≧200 mg/dL, and/or a LDL-C≧100 mg/dL on either no therapy or stable therapy. Any concomitant cholesterol medication must be at a stable dose for at least 2 months prior to baseline laboratory and not listed in the exclusion criteria. Cholesterol medications may include, but are not limited to, statins, niacin, ezetimibe, bile acid sequestrants (e.g., Welchol), fish oils, and combinations thereof. Useful statin compounds include, but are not limited to, atorvastatin, cerivastatin, fluvastatin, lovastatin, mevastatin, pitavastatin, pravastatin, rosuvastatin, simvastatin, and derivatives, salts, solvates, and prodrugs thereof. Treatment and dosage levels in statin therapy are well understood, and the statin can be provided in daily dosages of from about 1 mg to about 150 mg, in particular in a human patient. Exclusion criteria: patients with significant hepatic, renal or gastrointestinal tract disease, receiving thiazolidinediones or fibric acid derivatives, current overt cardiovascular disease, women of reproductive potential not receiving birth control, and pregnant/nursing women.

Study Design

The study provides the opportunity to evaluate a method for treatment of dyslipidemias. In other embodiments, the study can provide a method for lowering lipid levels or treating other metabolic disorders. In other embodiments, the study can provide a method for treating oxidative stress, or treating neurodegenerative disorders associated with oxidative stress. It utilizes a variety of endpoints and outcomes from analysis of molecular markers to standard clinical assessment. This design allows for an economy of effort, testing the putative agent and test combinations first clinically, with associated key endpoint biomarkers for which valuable validation data can be obtained.

Evaluation Criteria for clinical measurement: 1. Primary outcome measures: lipid laboratory markers (i.e., change in baseline TC, LDL-C, triglycerides (TG), HDL-C, non-HDL-C); 2. Secondary outcome measures: blood pressure (i.e., change in baseline systolic blood pressure (SBP) and/or diastolic blood pressure (DBP)); basic metabolic panel; AST (aspartate aminotransferase); ALT (alanine aminotransferase); and/or oxidative stress markers (i.e., change in baseline urine-derived markers of stress (e.g., isoprostanes: iPF2-Alpha-III/creat; iPF2-Alpha-VI/creat; 2,3 Dinor-iPF2-Alpha-III/creat)).

Subjects will be divided into four groups or arms: (1) high dose pterostilbene, 125 mg twice daily by mouth for 6 to 8 weeks; (2) low dose pterostilbene, 50 mg twice daily by mouth for 6 to 8 weeks; (3) low dose pterostilbene, 50 mg, in combination with ShanStar™ Concord Grape Extract, 100 mg, twice daily by mouth for 6 to 8 weeks; (4) matching placebo taken twice daily by mouth for 6 to 8 weeks; either one hour before or two hours after a meal. The pterostilbene will be standardized by ChromaDex, Inc.; the grape extract will be standardized by the Star PhytoNutrients Division of Cott Beverages, Inc., and combined with pterostilbene by ChromaDex, Inc. All products will be similar in appearance. Blood and urine will be collected at enrollment and final study visits. If the patient's LDL-C or TC is not within the inclusion criteria based on blood drawn at enrollment, the patient will not be allowed to initiate study medication. Patients will be actively participating for 6 to 8 weeks. Patients will be asked to monitor for any symptomatic adverse effects and home blood pressures, as needed. All study visits will consist of brief clinical examination (including vital signs), completed questionnaire (if appropriate), subjective adverse event reporting, and fasting donated blood and urine for clinical laboratory tests. At the enrollment visit, standard recommendations for therapeutic lifestyle interventions will be given to all groups, for example, provided in a printed handout. All blood clinical laboratory tests will be performed at an on-site laboratory. All urine clinical laboratory tests will be performed by an off-site laboratory specializing in oxidative stress analysis. At least 4 mL of urine will be collected during visits, in the morning while the patient is fasting. All policies and standard safeguards for decreasing urine contamination will be followed. Urine samples will be transferred from the collecting laboratory to a −80° C. freezer within 3 days of collection. Urine samples will then be shipped frozen on dry ice for analysis. Pill counts will be performed for each study subject to assess compliance.

In the above study design, there are no known food effects to be avoided. Without being bound by theory, it is known in the art ingestion of food can interfere with absorption of polyphenolic compounds in the body. It is expected that the study will take into account this food effect during administration of pterostilbene, or the combination of pterostilbene and a polyphenolic-PVP extract of grape juice.

Useful detection methods for the isoprostane stress markers listed above are available from Kronos Science (Phoenix, Ariz.) utilizing LC/MS/MS methodology to measure isoprostanes in urine and serum.

Isoprostanes are prostaglandin-like compounds formed from the free radical-catalyzed peroxidation of essential fatty acids (primarily arachidonic acid) without the direct action of cyclooxygenase (COX) enzyme. Isoprostanes are non-classical eicosanoids and possess potent biological activity as inflammatory mediators that augment the perception of pain. Isoprostanes are accurate markers of lipid peroxidation in both animal and human models of oxidative stress. For example, when there is an excessive production of lipid peroxidation products, which may be involved in the development or exacerbation of cancer, isoprostane analysis may measure this process. Isoprostanes may be used in this manner for cardiovascular and neurological diseases as well. Although isoprostanes have a short half-life, some of them have potent biological activities, especially in the lungs and kidney, and may even function in normal physiology. Isoprostanes are useful markers for oxidative stress, and importantly they can be assayed by non-invasive means.

Isoprostanes have been detected in all biological fluids and tissues analyzed to date. There is growing acceptance that measurement of the relatively stable F2-isoprostanes, and 8-IsoPF2α (iPF2-alpha-III) in urine is a reliable non-invasive approach to the determination of the degree of oxidative stress in patients. Normal levels of isoprostanes in healthy humans have been defined, so that the effects of disease states and subsequent therapeutic intervention can be determined. Thus, increased levels of urinary isoprostanes have been measured in many conditions that have been associated with excessive generation of free radicals, including poisoning with carbon tetrachloride, smoking, alcoholism, cirrhosis of the liver, brain degeneration, ischemia-reperfusion injury, atherosclerosis and diabetes. Urinary isoprostane analysis has also been used to assess the efficacy of antioxidants in vivo and to establish the value of antioxidant administration in clinical trials.

The results of the clinical study are summarized in Table 1 below. Data provided consisted of a pre-post study of 4 treatment groups including Placebo, Low Dose pterostilbene (LD), Low Dose pterostilbene plus a Grape extract (LD+Grape) and High Dose pterostilbene (HD), conducted on 20 participants per group (total N=80) on a variety of outcome measures. See Example 1 below.

Linear Mixed Models were used to estimate treatment effects in order to account for within-subject associations arising from the repeated measures pre-post longitudinal design. Loss to follow up was minimal (<10%) and the underlying Missing At Random architecture implicit in mixed models was assumed. Various models were fit to examine potential baseline effects including as appropriate: 1) 3-way interaction models of final outcome×treatment group×baseline value; 2) 2-way interaction models including all 2-way terms from (1) but excluding the 3-way term; 3) models assuming baseline value affected change similarly across treatment groups; 4) models assuming change in outcome was independent of baseline value.

Each model was examined in unadjusted and adjusted form (adjusting for age, sex and race: White, Black, & Asian/Other). Final reported treatment effects were obtained from the simplest appropriate adjusted model for each outcome; in all but 2 cases (Weight, body mass index (BMI)), change appeared potentially related to the baseline values but not differentially across treatment group (model 3). For Weight & BMI outcomes, baseline value was not supported as affecting change in this data.

Adjusted Treatment Effects (differences in outcome changes comparing treated versus placebo groups) are summarized as follows.

TABLE 1
Adjusted Differences in Expected Outcomes changes Compared to Placebo
(Treament Effects)

| | | Treatment Group | | |
|---|---|---|---|---|
| Outcome | | LD | LD + Grape | HD |
| 1 | SBP | −3.67 | −6.72 | −7.77 |
| | (mm Hg) | p = 0.184 | p = 0.016 | p = 0.005 |
| | | (−9.09, 1.74) | (−12.18, −1.25) | (−13.17, −2.38) |
| 2 | DBP | −2.23 | −3.31 | −7.32 |
| | (mm Hg) | p = 0.322 | p = 0.146 | p = 0.001 |
| | | (−6.64, 2.18) | (−7.77, 1.15) | (−11.72, −2.92) |
| 3 | LDL | 20.04 | 5.33 | 19.67 |
| | (mg/dL) | p = 0.006 | p = 0.471 | p = 0.007 |
| | | (5.68, 34.40) | (−9.16, 19.82) | (5.28, 34.05) |
| 4 | HDL | −2.89 | −0.83 | −3.28 |
| | (mg/dL) | p = 0.130 | p = 0.660 | p = 0.081 |
| | | (−6.62, 0.85) | (−4.55, 2.88) | (−6.97, 0.41) |
| 5 | TG | 0.77 | −2.91 | 1.52 |
| | (mg/dL) | p = 0.949 | p = 0.816 | p = 0.901 |
| | | (−23.07, 24.61) | (−27.47, 21.65) | (−22.32, 25.35) |
| 6 | TC | 18.10 | 4.56 | 16.39 |
| | (mg/dL) | p = 0.026 | p = 0.578 | p = 0.043 |
| | | (2.19, 34.00) | (−11.50, 20.63) | (0.49, 32.30) |
| 7 | Weight | −3.04 | −1.27 | −1.75 |
| | (lb.) | p = 0.055 | p = 0.430 | p = 0.269 |
| | | (−6.14, 0.06) | (−4.41, 1.88) | (−4.85, 1.35) |
| 8 | BMI | −0.45 | −0.22 | −0.27 |
| | (kg/m²) | p = 0.059 | p = 0.368 | p = 0.268 |
| | | (−0.92, 0.02) | (−0.70, 0.26) | (−0.74, 0.20) |
| 9 | AST | 1.86 | 0.12 | 0.94 |
| | | p = 0.220 | p = 0.938 | p = 0.541 |
| | | (−1.11, 4.84) | (−2.87, 3.11) | (−2.07, 3.95) |
| 10 | ALT | 1.60 | −1.21 | −0.03 |
| | | p = 0.400 | p = 0.529 | p = 0.989 |
| | | (−2.13, 5.33) | (−4.98, 2.56) | (−3.77, 3.71) |
| 11 | BG | −3.35 | −1.25 | 0.76 |
| | | p = 0.263 | p = 0.685 | p = 0.802 |
| | | (−9.22, 2.52) | (−7.26, 4.77) | (−5.16, 6.68) |
| 12 | SCR | −0.01 | 0.00 | −0.03 |
| | | p = 0.666 | p = 0.864 | p = 0.135 |
| | | (−0.05, 0.03) | (−0.04, 0.04) | (−0.07, 0.01) |

BG: blood glucose (mg/dL); SCR: serum creatinine (mg/dL); AST, ALT (units/L)

The methods described above may be further understood in connection with the following Examples. As used herein, the term “HPMC” means hydroxypropyl methylcellulose.

### EXAMPLE 1

In accordance with one embodiment, a patient (or each member of a first clinical trial patient group) was treated with a daily dosage as follows: 100 mg pterostilbene combined with 200 mg ShanStar™ Concord Grape Extract powder formulated in an acceptable pharmaceutical carrier, administered orally as follows. 50 mg pterostilbene, 100 mg ShanStar™ Concord Grape Extract powder, and 25 mg microcrystalline cellulose were combined in a green opaque size 2 HPMC capsule, and administered twice daily.

In accordance with the three other clinical trial patient groups, pterostilbene was administered orally as follows as either a high daily dose (250 mg) and a low daily dose (100 mg). High dose: 125 mg pterostilbene and 35 mg microcrystalline cellulose were combined in a green opaque size 2 HPMC capsule, and administered twice daily. Low dose: 50 mg pterostilbene and 120 mg microcrystalline cellulose were combined in a green opaque size 2 HPMC capsule, and administered twice daily. Placebo: 170 mg microcrystalline cellulose in a green opaque size 2 HPMC capsule, administered twice daily. Approximately 36% of all subjects, spread randomly across all patient groups, were undergoing concurrent cholesterol therapy (for at least about 2 months prior to baseline/initial measurement).

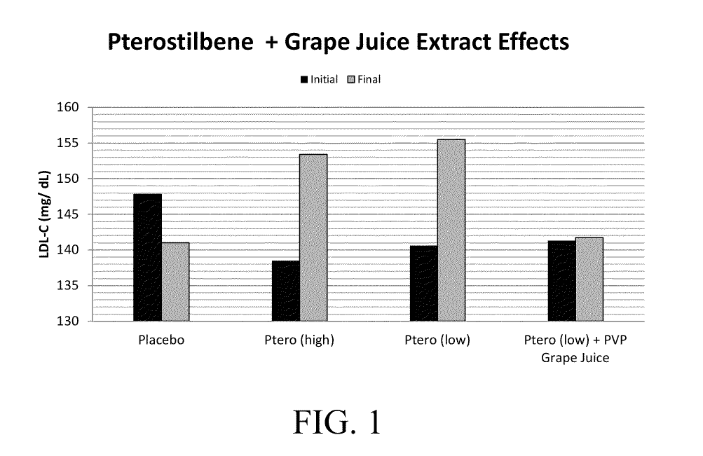

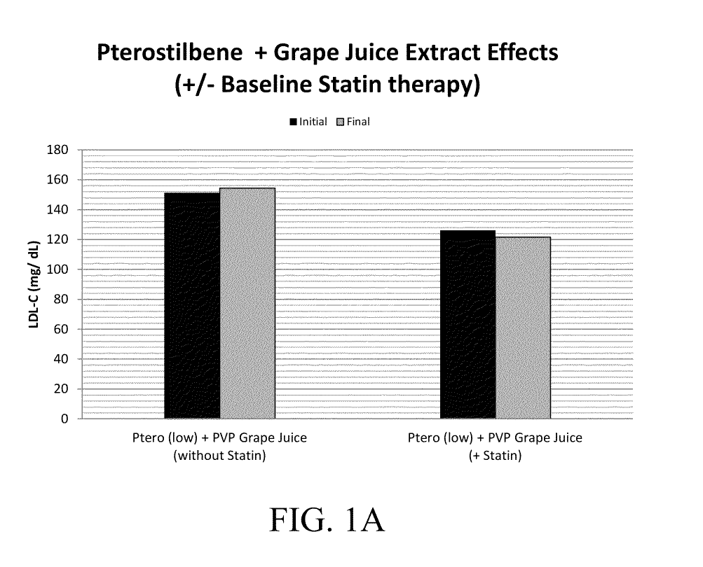

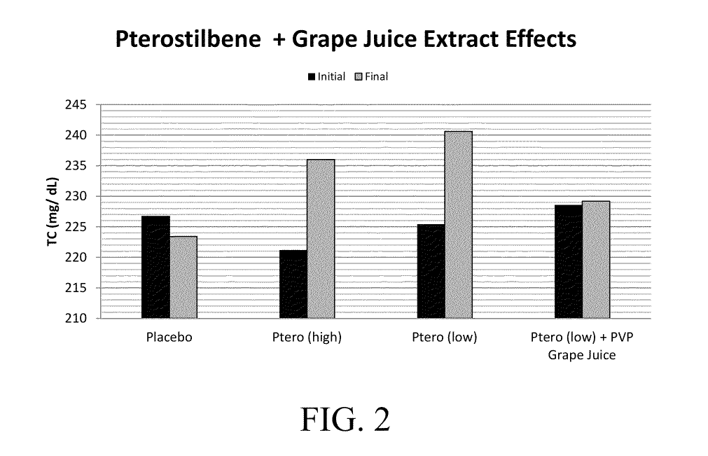

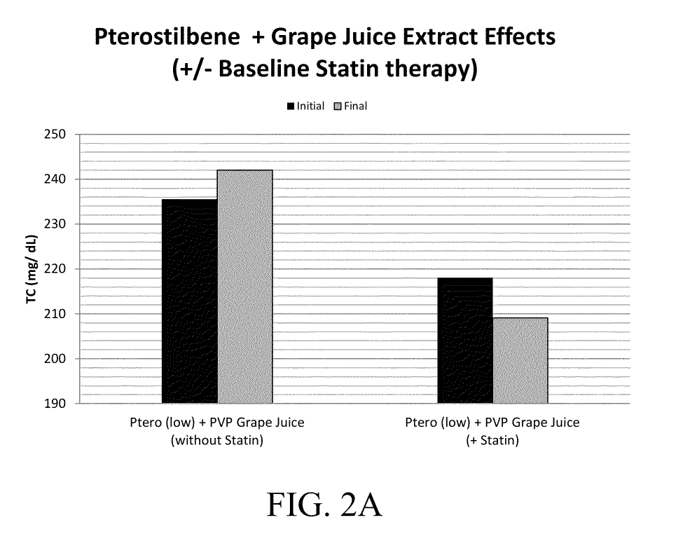

As shown in FIGS. 1 and 2, after 8 weeks of study monitoring (final measurement, 73/80 patients completed the study), administration of pterostilbene resulted in increases in TC and LDL-C levels in the patient study groups. At the low dose of pterostilbene, both TC and LDL-C increases were effectively attenuated when combined with grape extract (including those patients receiving cholesterol medication). Stated in another way, presence of a baseline cholesterol medication as statin therapy appeared to attenuate LDL-raising effects as well (FIGS. 1A, 2A). Thus, it was demonstrated that human subjects exhibited relative lowering of lipid levels in plasma (TC and/or LDL-C), as tested in the patient's blood, to a greater extent than subjects administered pterostilbene alone.

### EXAMPLE 1A

In the same study, patients administered the high dose of pterostilbene exhibited reductions in systolic blood pressure (−7.8 mmHg; p<0.01) and diastolic blood pressure (−7.3 mmHg; p<0.001), including patients receiving cholesterol medication. Patients administered the low dose of pterostilbene exhibited reductions in systolic blood pressure (−3.7 mmHg; p<0.18) and diastolic blood pressure (−2.2 mmHg; p<0.32), including patients receiving cholesterol medication. Patients administered the low dose of pterostilbene combined with grape extract exhibited reductions in systolic blood pressure (−6.7 mmHg; p<0.02) and diastolic blood pressure (−3.3 mmHg; p<0.15), including patients receiving cholesterol medication.

Thus, it was demonstrated that pterostilbene, alone and in combination with the grape extract, lowered blood pressure in adult patients compared to placebo.

It is expected that pterostilbene, when administered at the higher dose, in combination with the grape extract, will lower blood pressure in adult patients compared to placebo. It is further expected that pterostilbene, when administered at the higher dose, in combination with the grape extract, will lower blood pressure in hypertensive patients compared to placebo.

### EXAMPLE 2

In accordance with one embodiment, a patient (or each member of a patient group) is treated with a daily dosage as follows: 150 mg pterostilbene combined with 300 mg ShanStar™ Concord Grape Extract powder formulated in an acceptable pharmaceutical carrier, administered orally as in Example 1. After about 8 weeks of study monitoring, it is expected that an individual human subject will exhibit lowering of lipid levels in plasma (TC and/or LDL-C), as tested in the patient's blood, to a greater extent than a patient administered pterostilbene alone.

### EXAMPLE 3

Example 1 is repeated. After about 8 weeks of study monitoring, it is expected that an individual human subject administered pterostilbene in combination with the grape extract will exhibit lowering of oxidative stress markers, as tested in the patient's urine, to a greater extent than a patient administered pterostilbene alone.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A lipid-lowering composition comprising a therapeutically effective amount of pterostilbene, a therapeutically effective amount of a polyphenolic-PVP extract of grape juice, and a pharmaceutically acceptable carrier.

2. The lipid-lowering composition of claim 1, wherein pterostilbene is present in an amount from about 10 mg to about 500 mg, and the polyphenolic-PVP extract of grape juice is present in an amount from about 100 mg to about 300 mg.

3. The lipid-lowering composition of claim 1, wherein pterostilbene is present in an amount from about 25 mg to about 500 mg, and the polyphenolic-PVP extract of grape juice is present in an amount from about 100 mg to about 300 mg.

4. The lipid-lowering composition of claim 1, wherein pterostilbene is present in an amount from about 50 mg to about 150 mg.

5. An antiinflammatory composition comprising a therapeutically effective amount of pterostilbene, a therapeutically effective amount of a polyphenolic-PVP extract of grape juice, and a pharmaceutically acceptable carrier.

6. The antiinflammatory composition of claim 5, wherein pterostilbene is present in an amount from about 10 mg to about 500 mg, and the polyphenolic-PVP extract of grape juice is present in an amount from about 100 mg to about 300 mg.

7. The antiinflammatory composition of claim 5, wherein pterostilbene is present in an amount from about 25 mg to about 500 mg, and the polyphenolic-PVP extract of grape juice is present in an amount from about 100 mg to about 300 mg.

8. The antiinflammatory composition of claim 5, wherein pterostilbene is present in an amount from about 50 mg to about 150 mg.

9. A method of lowering lipid levels in an individual, comprising administering to the individual in need of such treatment a composition comprising a therapeutically effective amount of pterostilbene, a therapeutically effective amount of a polyphenolic-PVP extract of grape juice, and a pharmaceutically acceptable carrier, wherein lipid levels are decreased compared to the individual administered pterostilbene alone.

10. The method of claim 9, wherein the therapeutically effective amount of pterostilbene for a total daily dose is in a range of about 10 mg to about 500 mg, and the therapeutically effective amount of the polyphenolic-PVP extract of grape juice for a total daily dose is in a range of about 100 mg to about 300 mg.

11. The method of claim 9, wherein the therapeutically effective amount of pterostilbene for a total daily dose is in a range of about 50 mg to about 250 mg, and the therapeutically effective amount of the polyphenolic-PVP extract of grape juice for a total daily dose is in a range of about 100 mg to about 300 mg.

12. The method of claim 9, wherein the decreased lipid levels are expressed as a reduction in blood serum selected from the group consisting of total cholesterol (TC), low-density lipoprotein cholesterol (LDL-C), triglycerides (TG), and the ratio of low-density lipoprotein cholesterol to high-density lipoprotein cholesterol (LDL-C/HDL-C).

13. The method of claim 10, wherein the individual is a human.

14. The method of claim 12, wherein the individual is a human.

15. A method of lowering blood pressure, comprising administering to the individual in need of such treatment a composition comprising a therapeutically effective amount of pterostilbene, a therapeutically effective amount of a polyphenolic-PVP extract of grape juice, and a pharmaceutically acceptable carrier, wherein systolic blood pressure (SBP) or diastolic blood pressure (DBP) is decreased.

16. The method of claim 15, wherein the therapeutically effective amount of pterostilbene for a total daily dose is in a range of about 10 mg to about 500 mg, and the therapeutically effective amount of the polyphenolic-PVP extract of grape juice for a total daily dose is in a range of about 100 mg to about 300 mg.

17. The method of claim 15, wherein the therapeutically effective amount of pterostilbene for a total daily dose is in a range of about 50 mg to about 250 mg, and the therapeutically effective amount of the polyphenolic-PVP extract of grape juice for a total daily dose is in a range of about 100 mg to about 300 mg.

18. The method of claim 16, wherein the individual is a human.

19. A method of lowering low-density lipoprotein cholesterol (LDL-C) and raising high-density lipoprotein cholesterol (HDL-C) in an individual, comprising administering to the individual in need of such treatment a composition comprising a therapeutically effective amount of pterostilbene, a therapeutically effective amount of a polyphenolic-PVP extract of grape juice, and a pharmaceutically acceptable carrier, wherein (LDL-C) levels are decreased, and wherein (HDL-C) levels are increased.

20. The method of claim 19, wherein the therapeutically effective amount of pterostilbene for a total daily dose is in a range of about 10 mg to about 500 mg, and the therapeutically effective amount of the polyphenolic-PVP extract of grape juice for a total daily dose is in a range of about 100 mg to about 300 mg.

21. The method of claim 20, wherein the individual is a human.
